SEQUENCE LISTING (8 sequences)

SEQ ID NO: 1 - DNA, 22 bp, Artificial sequence
  Synthetic sense primer
ctgagtatgtcgtggagtctac

SEQ ID NO: 2 - DNA, 22 bp, Artificial sequence
  Synthetic antisense primer
gttacgtaggacgtggtggttg

SEQ ID NO: 3 - DNA, 20 bp, Artificial sequence
  Synthetic sense primer
acggcatggatctcaaagac

SEQ ID NO: 4 - DNA, 17 bp, Artificial sequence
  Synthetic antisense primer
tgatgcacgagtgggtg

SEQ ID NO: 5 - DNA, 20 bp, Artificial sequence
  Synthetic sense primer
ttgcttcagctccacagaga

SEQ ID NO: 6 - DNA, 20 bp, Artificial sequence
  Synthetic antisense primer
caggaacgggagatcttggt

SEQ ID NO: 7 - DNA, 20 bp, Artificial sequence
  Synthetic sense primer
acgtcctggtgaagttggtc

SEQ ID NO: 8 - DNA, 20 bp, Artificial sequence
  Synthetic antisense primer
tcctctttctccgaagggac

(19) United States
(12) Patent Application Publication
(10) Pub. No.: US 2008/0119439 A1
(43) Pub. Date: May 22, 2008

(54) Treatment and prevention of intestinal fibrosis

(75) Inventors: John Parkinson, Martinez, CA (US); Stefano Fiorucci, Perugia (IT)

Correspondence Address: JACQUELINE S LARSON, 245 AVINGTON ROAD, ALAMEDA, CA, 94502, US

(73) Assignee: Bayer Schering Pharma Aktiengesellschaft

(21) Appl. No.: 11/895,027

(22) Filed: Aug. 21, 2007

Related U.S. Application Data
(60) Provisional application No. 60/839,755, filed on Aug. 23, 2006.

(51) Int. Cl. A61K 31/724; A61K 31/426; A61K 31/421; A61K 31/4166; A61K 31/385; A61K 31/365; A61K 31/202

(52) U.S. Cl. 514/58; 514/369; 514/376; 514/386; 514/440; 514/462; 514/463; 514/559

(57) ABSTRACT

The present invention is directed to the use of lipoxin A₄ analogs as therapeutic agents in the treatment and/or prevention of intestinal fibrosis.

This application also claims the benefit of and priority to U.S. Ser. No. 60/839,755 filed on Aug. 23, 2006, which application is hereby incorporated by reference in its entirety.

This invention relates to the use of lipoxin A₄ analogs as therapeutic agents in the treatment and/or prevention of intestinal fibrosis.

Intestinal disorders, such as inflammatory bowel diseases (IBDs) (including Crohn's disease and ulcerative colitis) and collagenous colitis are life-long, relapsing conditions affecting primarily the gastrointestinal tract of individuals. Fibrosis is a complication of such disorders. Current therapies can relieve inflammation but do not alter the natural history of the disease or its progression to intestinal fibrosis and obstruction that often results in bowel resection. Unfortunately, surgery does not prevent the recurrence of bowel inflammation or the ECM changes that cause fibrosis and stenosis.

Lipoxins, together with leukotrienes, prostaglandins, and thromboxanes, constitute a group of biologically active oxygenated fatty acids collectively referred to as the eicosanoids. Eicosanoids are all synthesized de novo from membrane phospholipid via the arachidonic acid cascade of enzymes. Since their initial discovery in 1984, it has become apparent that lipoxins, which are a structurally unique class of eicosanoids, possess potent anti-inflammatory properties that suggest they may have therapeutic potential (Serhan, C. N., Prostaglandins (1997), Vol. 53, pp. 107-137; O'Meara, Y. M. et al., Kidney Int. (Suppl.) (1997), Vol. 58, pp. S56-S61; Brady, H. R. et al., Curr. Opin. Nephrol. Hypertens. (1996), Vol. 5, pp. 20-27; and Serhan, C. N., Biochem. Biophys. Acta. (1994), Vol. 1212, pp. 1-25). Of particular interest is the ability of lipoxins to antagonise the pro-inflammatory functions of leukotrienes in addition to other inflammatory agents such as platelet activating factor, FMLP, immune complexes and TNFα. Lipoxins are thus potent anti-neutrophil (PMN) agents which inhibit PMN chemotaxis, homotypic aggregation, adhesion, migration across endothelial and epithelial cells, margination/diapedesis and tissue infiltration (Lee, T. H., et al., Clin. Sci. (1989), Vol. 77, pp. 195-203; Fiore, S., et al., Biochemistry (1995), Vol. 34, pp. 16678-16686; Papyianni, A., et al., J. Immunol. (1996), Vol. 56, pp. 2264-2272; Hedqvist, P., et al., Acta. Physiol. Scand. (1989), Vol. 137, pp. 157-572; Papyianni, A., et al., Kidney Intl. (1995), Vol. 47, pp. 1295-1302). In addition, lipoxins are able to down-regulate endothelial P-selectin expression and adhesiveness for PMNs (Papyianni, A., et al., J. Immunol. (1996), Vol. 56, pp. 2264-2272), bronchial and vascular smooth muscle contraction, mesangial cell contraction and adhesiveness (Dahlen, S. E., et al., Adv. Exp. Med. Biol. (1988), Vol. 229, pp. 107-130; Christie, P. E., et al., Am. Rev. Respir. Dis. (1992), Vol. 145, pp. 1281-1284; Badr, K. F., et al., Proc. Natl. Acad. Sci. (1989), Vol. 86, pp. 3438-3442; and Brady, H. R., et al., Am. J. Physiol. (1990), Vol. 259, pp. F809-F815) and eosinophil chemotaxis and degranulation (Soyombo, O., et al., Allergy (1994), Vol. 49, pp. 230-234).

This unique anti-inflammatory profile of lipoxins, particularly lipoxin A₄, has prompted interest in exploiting their potential as therapeutics for the treatment of inflammatory or autoimmune disorders and pulmonary and respiratory tract inflammation.

As with other endogenous eicosanoids, naturally-occurring lipoxins are unstable products which are rapidly metabolized and inactivated (Serhan, C. N., Prostaglandins (1997), Vol. 53, pp. 107-137). This has limited the development of the lipoxin field of research, particularly with respect to in vivo pharmacological assessment of the anti-inflammatory profile of lipoxins. Several U.S. patents have issued directed to compounds having the active site of lipoxin A₄, but with a longer tissue half-life. See, e.g., U.S. Pat. Nos. 5,441,951 and 5,648,512, the disclosures of which are incorporated in full by reference herein. These compounds retain lipoxin A₄ receptor binding activity and the potent in vitro and in vivo anti-inflammatory properties of natural lipoxins (Takano, T., et al., J. Clin. Invest. (1998), Vol. 101, pp. 819-826; Scalia, R., et al., Proc. Natl. Acad. Sci. (1997), Vol. 94, pp. 9967-9972; Takano, T., et al., J. Exp. Med. (1997), Vol. 185, pp. 1693-1704; Maddox, J. F., et al., J. Biol. Chem. (1997), Vol. 272, pp. 6972-6978; Serhan, C. N., et al., Biochemistry (1995), Vol. 34, pp. 14609-14615).

The present invention is directed to the use of certain agents as therapeutics for the treatment or prevention of intestinal fibrosis. More particularly, the invention is directed to a method of treating or preventing intestinal fibrosis by administering to a patient in need thereof a therapeutically effective amount of a lipoxin A₄ analog.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the singular forms “a”, “an”, and “the” include plural referents unless the context clearly dictates otherwise. For example, “a compound” refers to one or more of such compounds, while “the enzyme” includes a particular enzyme as well as other family members and equivalents thereof as known to those skilled in the art.

Furthermore, as used in the specification and appended claims, unless specified to the contrary, the following terms have the meaning indicated:

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by one or more substituents selected from the group consisting of cyano, nitro, —R⁹—OR⁶, —R⁹—N═N—O—R¹⁶, —R⁹—N(R⁶)₂, —R⁹—C(O)R⁶, —R⁹—C(O)OR⁶, —R⁹—C(O)N(R⁶)₂, —R⁹—N(R⁶)C(O)OR¹⁶, —R⁹—N(R⁶)C(O)R⁶, —R⁹—S(O)ₜOR⁶ (where t is 0 to 2), —R⁹—S(O)ₜOR⁶ (where t is 0 to 2), —R⁹—S(O)ₜN(R⁶)₂ (where t is 0 to 2) where each R⁶ and R⁹ is as defined above in the Summary of the Invention and each R¹⁶ is hydrogen, alkyl or aralkyl. Unless stated otherwise specifically in the specification, it is understood that such substitution can occur on any carbon of the alkyl group.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like.

“Alkenyl” refers to a straight or branched monovalent hydrocarbon chain radical consisting solely of carbon and hydrogen atoms, containing at least one double bond, having from two to eight carbon atoms, and which is attached to the rest of the molecule by a single bond, e.g., ethenyl, prop-1-enyl, but-1-enyl, pent-1-enyl, penta-1,4-dienyl, and the like. Unless stated otherwise specifically in the specification, the alkenyl radical may be optionally substituted by one or more substituents selected from the group consisting of cyano, nitro, —R⁹—OR⁶, —R⁹—N═N—O—R¹⁶, —R⁹—N(R⁶)₂, —R⁹—C(O)R⁶, —R⁹—C(O)OR⁶, —R⁹—C(O)N(R⁶)₂, —R⁹—N(R⁶)C(O)OR¹⁶, —R⁹—N(R⁶)C(O)R⁶, —R⁹—S(O)ₜOR⁶ (where t is 0 to 2), —R⁹—S(O)ₜOR⁶ (where t is 0 to 2), —R⁹—S(O)ₜN(R⁶)₂ (where t is 0 to 2) where each R⁶ and R⁹ is as defined above in the Summary of the Invention and each R¹⁶ is hydrogen, alkyl or aralkyl. Unless stated otherwise specifically in the specification, it is understood that such substitution can occur on any carbon of the alkenyl group.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like.

“Alkynyl” refers to a straight or branched monovalent hydrocarbon chain radical consisting solely of carbon and hydrogen atoms, containing at least one triple bond, having from two to eight carbon atoms, and which is attached to the rest of the molecule by a single bond, e.g., ethynyl, prop-1-ynyl, but-1-ynyl, pent-1-ynyl, pent-3-ynyl, and the like. Unless stated otherwise specifically in the specification, the alkynyl radical may be optionally substituted by one or more substituents selected from the group consisting of cyano, nitro, —R⁹—OR⁶, —R⁹—N═N—O—R¹⁶, —R⁹—N(R⁶)₂, —R⁹—C(O)R⁶, —R⁹—C(O)OR⁶, —R⁹—C(O)N(R⁶)₂, —R⁹—N(R⁶)C(O)OR¹⁶, —R⁹—N(R⁶)C(O)R⁶, —R⁹—S(O)ₜOR⁶ (where t is 0 to 2), —R⁹—S(O)ₜR⁶ (where t is 0 to 2), —R⁹—S(O)ₜN(R⁶)₂ (where t is 0 to 2) where each R⁶ and R⁹ is as defined above in the Summary of the Invention and each R¹⁶ is hydrogen, alkyl or aralkyl. Unless stated otherwise specifically in the specification, it is understood that for radicals, as defined below, that contain a substituted alkynyl group that the substitution can occur on any carbon of the alkynyl group.

“Alkynylene chain” refers to a straight or branched divalent hydrocarbon chain consisting solely of carbon and hydrogen, containing at least one triple bond and having from two to eight carbon atoms, e.g., ethynylene, prop-1-ynylene, but-1-ynylene, pent-3-ynylene, hexa-1,4-diynylene, and the like.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like.

“Amino” refers to the —NH₂ radical.

“Aryl” refers to a phenyl or naphthyl radical. Unless stated otherwise specifically in the specification, the term “aryl” or the prefix “ar-” (such as in “aralkyl”) is meant to include aryl radicals which may be optionally substituted by one or more substituents selected from the group consisting of alkyl, alkenyl, halo, haloalkyl, cyano, nitro, aryl, aralkyl, cycloalkyl, —R⁹—OR⁶, —R⁹—N═N—O—R¹⁶, —R⁹—N(R⁶)₂, —R⁹—C(O)R⁶, —R⁹—C(O)OR⁶, —R⁹—C(O)N(R⁶)₂, —R⁹—N(R⁶)C(O)OR¹⁶, —R⁹—N(R⁶)C(O)R⁶, —R⁹—S(O)ₜOR⁶ (where t is 0 to 2), —R⁹—S(O)ₜR⁶ (where t is 0 to 2), —R⁹—S(O)ₜN(R⁶)₂ (where t is 0 to 2) where each R⁶ and R⁹ is as defined above in the Summary of the Invention and each R¹⁶ is hydrogen, alkyl or aralkyl. Unless stated otherwise specifically in the specification, it is understood that such substitution can occur on any carbon of the aryl group.

“Aralkyl” refers to a radical of the formula —RₐR_{b} where Rₐ is an alkyl radical as defined above and R_{b} is an aryl radical as defined above, e.g., benzyl, and the like. The aryl radical may be optionally substituted as described above.

“Carboxy” refers to the —C(O)OH radical.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

“Clathrates” as used herein refers to substances which fix gases, liquids or compounds as inclusion complexes so that the complex may be handled in solid form and the included constituent (or “guest” molecule) subsequently releases by the action of a solvent or by melting. The term “clathrate” is used interchangeably herein with the phrase “inclusion molecule” or with the phrase “inclusion complex”. Clathrates used in the instant invention are prepared from cyclodextrins. Cyclodextrins are widely known as having the ability to form clathrates (i.e., inclusion compounds) with a variety of molecules. See, for example, Inclusion Compounds, edited by J. L. Atwood, J. E. D. Davies, and D. D. MacNicol, London, Orlando, Academic Press, 1984; Goldberg, I., “The Significance of Molecular Type, Shape and Complementarity in Clathrate Inclusion”, Topics in Current Chemistry (1988), Vol. 149, pp. 244; Weber, E. et al., “Functional Group Assisted Clathrate Formation—Scissor-Like and Roof-Shaped Host Molecules”, Topics in Current Chemistry (1988), Vol. 149, pp. 45-135; and MacNicol, D. D. et al., “Clathrates and Molecular Inclusion Phenomena”, Chemical Society Reviews (1978), Vol. 7, No. 1, pp. 65-87. Conversion into cyclodextrin clathrates is known to increase the stability and solubility of certain compounds, thereby facilitating their use as pharmaceutical agents. See, for example, Saenger, W., “Cyclodextrin Inclusion Compounds in Research and Industry”, Angew. Chem. Int. Ed. Engl. (1980), Vol. 19, pp. 344-362; U.S. Pat. No. 4,886,788 (Schering AG); U.S. Pat. No. 6,355,627 (Takasago); U.S. Pat. No. 6,288,119 (Ono Pharmaceuticals); U.S. Pat. No. 6,110,969 (Ono Pharmaceuticals); U.S. Pat. No. 6,235,780 (Ono Pharmaceuticals); U.S. Pat. No. 6,262,293 (Ono Pharmaceuticals); U.S. Pat. No. 6,225,347 (Ono Pharmaceuticals); and U.S. Pat. No. 4,935,446 (Ono Pharmaceuticals).

“Cyclodextrin” refers to cyclic oligosaccharides consisting of at least six glucopyranose units which are joined together by α(1-4) linkages. The oligosaccharide ring forms a torus with the primary hydroxyl groups of the glucose residues lying on the narrow end of the torus. The secondary glucopyranose hydroxyl groups are located on the wider end. Cyclodextrins have been shown to form inclusion complexes with hydrophobic molecules in aqueous solutions by binding the molecules into their cavities. The formation of such complexes protects the “guest” molecule from loss of evaporation, from attack by oxygen, visible and ultraviolet light and from intra- and intermolecular reactions. Such complexes also serve to “fix” a volatile material until the complex encounters a warm moist environment, at which point the complex will dissolve and dissociate into the guest molecule and the cyclodextrin. For purposes of this invention, the six-glucose unit containing cyclodextrin is specified as α-cyclodextrin, while the cyclodextrins with seven and eight glucose residues are designated as β-cyclodextrin and γ-cyclodextrin, respectively. The most common alternative to the cyclodextrin nomenclature is the naming of these compounds as cycloamyloses.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, aryl, aralkyl, cycloalkyl, heterocyclyl, heterocyclylalkyl, —R⁹—OR⁶, —R⁹—N═N—O—R¹⁶, —R⁹—N(R⁶)₂, —R⁹—C(O)R⁶, —R⁹—C(O)OR⁶, —R⁹—C(O)N(R⁶)₂, —R⁹—N(R⁶)C(O)OR¹⁶, —R⁹—N(R⁶)C(O)R⁶, —R⁹—S(O)ₜOR⁶ (where t is 0 to 2), —R⁹—S(O)ₜR⁶ (where t is 0 to 2), —R⁹—S(O)ₜN(R⁶)₂ (where t is 0 to 2) where each R⁶ and R⁹ is as defined above in the Summary of the Invention and each R¹⁶ is hydrogen, alkyl or aralkyl. Unless stated otherwise specifically in the specification, it is understood that such substitution can occur on any carbon of the cycloalkyl group.

“Cycloalkylene” refers to a stable divalent monocyclic or bicyclic hydrocarbon consisting solely of carbon and hydrogen atoms, having from three to ten carbon atoms, and which is saturated and attached to the rest of the molecule by two single bonds, e.g., cyclopropylene, cyclobutylene, cyclopentylene, cyclohexylene, decalinylene and the like. Unless otherwise stated specifically in the specification, the term “cycloalkylene” is meant to include cycloalkylene moieties which are optionally substituted by one or more substituents independently selected from the group consisting of alkyl, alkoxy, halo, haloalkyl, haloalkoxy, hydroxy, amino, and carboxy.

“Halo” refers to bromo, chloro, iodo or fluoro.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Mammal” includes humans and domesticated animals, such as cats, dogs, swine, cattle, sheep, goats, horses, rabbits, and the like. Preferably, for purposes of this invention, the mammal is a human.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Stable compound” and “stable structure” are meant to indicate a compound that is sufficiently robust to survive isolation to a useful degree of purity from a reaction mixture, and formulation into an efficacious therapeutic agent.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the invention and a medium generally accepted in the art for the delivery of the biologically active compound to mammals, for example, humans. Such a medium includes all pharmaceutically acceptable carriers, diluents or excipients therefore, all of which are encompassed herein within the term “pharmaceutically acceptable excipient”.

“Therapeutically effective amount” refers to that amount of a compound of the invention which, when administered to a mammal, preferably a human, is sufficient to effect treatment, as defined below, or prevention of a disease or condition of interest in the mammal, preferably a human. The amount of a compound of the invention which constitutes a “therapeutically effective amount” will vary depending on the compound, the disease or condition and its severity, the age of the mammal to be treated, and other known and quantifiable variables, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) stabilizing the disease or condition.

The compounds of the invention, as a single stereoisomer, a mixture of stereoisomers, or as a racemic mixture of stereoisomers; or as a cyclodextrin clathrate thereof, or as a pharmaceutically acceptable salt thereof, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

Lipoxin A₄ analogs useful in the present invention can be selected from those disclosed and claimed in U.S. Pat. No. 6,831,186 and Pat. publication No. US2004-0162433, the entire disclosures of which are incorporated herein by reference. These compounds are potent, selective and metabolically/chemically stable lipoxin A₄ analogs useful in treating inflammatory or autoimmune disorders and pulmonary or respiratory tract inflammation in mammals, particularly humans. The production of these compounds and the pharmaceutically acceptable salts thereof are described in detail in the above US patent and publication. The above lipoxin A₄ analogs have not been previously disclosed as being effective in the treatment or prevention of intestinal fibrosis.

Thus, in one aspect, the invention is directed to the use of lipoxin A₄ analog compounds of formula (I) or formula (II) for the treatment and/or prevention of intestinal fibrosis:

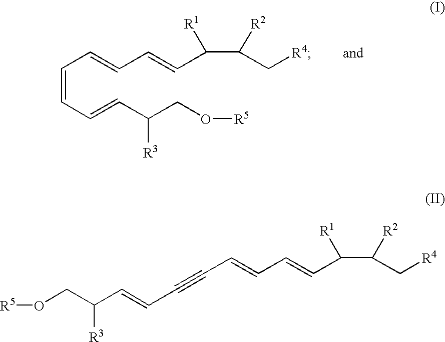

wherein:
each R¹, R² and R³ are independently halo, —OR⁶, —SR⁶, —S(O)ₜR⁷ (where t is 1 or 2) or —N(R⁷)R⁸;
or R¹ and R² together with the carbons to which they are attached form a monocyclic heterocyclic structure selected from the following:

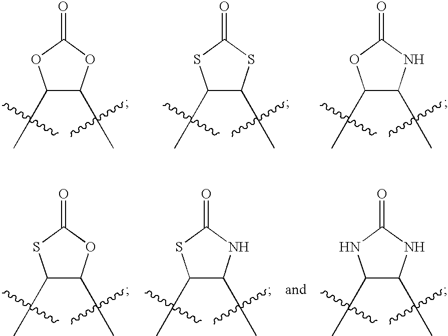

or R¹ and R² together with the carbons to which they are attached form the following bicyclic heterocyclic structure:

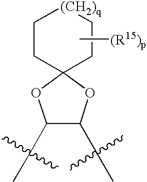

(where q is 0 to 3, p is 1 to 4 and each R¹⁵ is hydrogen, alkyl, aralkyl or aryl);
each R⁴ is —R⁹—R¹², —R⁹—R¹³—R¹¹, —R⁹—O—R¹⁰—R¹¹, —R⁹—O—R¹², —R⁹—C(O)—R¹⁰—R¹¹, —R⁹—N(R⁷)—R¹⁰—R¹¹, —R⁹—S(O)ₜ—R¹⁰—R¹¹ (where t is 0 to 2), or —R⁹—C(F)₂—R⁹—R¹¹;
each R⁵ is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
each R⁶ is independently hydrogen, alkyl, aryl, aralkyl, —C(O)R⁷, —C(S)R⁷, —C(O)OR¹⁴, —C(S)OR¹⁴, —C(O)N(R⁷)R⁸, or —C(S)N(R⁷)R⁸;
each R⁷ is independently hydrogen, alkyl, cycloalkyl, aryl, or aralkyl;
R⁸ is independently hydrogen, alkyl, aryl, aralkyl, —C(O)R⁷, —C(O)OR¹⁴, or cycloalkyl (optionally substituted with one more substituents selected from alkyl, —N(R⁷)₂, and —C(O)OR⁷);
each R⁹ is independently a direct bond or a straight or branched alkylene chain;
each R¹⁰ is independently a straight or branched alkylene chain, a straight or branched alkenylene chain, a straight or branched alkynylene chain or a cycloalkylene;
each R¹¹ is independently —C(O)OR⁷, —C(O)N(R⁷)₂, —P(O)(OR⁷)₂, —S(O)₂OR⁷, —S(O)₂N(H)R⁷ or tetrazole;
R¹² is aryl (substituted by —C(O)OR⁷ or —C(O)N(R⁷)₂ and optionally by one or more substituents selected from alkyl, alkoxy, halo, haloalkyl and haloalkoxy) or aralkyl (substituted by —C(O)OR⁷ or —C(O)N(R⁷)₂ and optionally by one or more substituents selected from alkyl, alkoxy, halo, haloalkyl and haloalkoxy);
R¹³ is a branched alkylene chain, a straight or branched alkenylene chain or a cycloalkylene; and
R¹⁴ is alkyl, aryl or aralkyl;
   as a single stereoisomer, a mixture of stereoisomers, or a racemic mixture of stereoisomers;
   or as a cyclodextrin clathrate thereof, or as a pharmaceutically acceptable salt thereof.

In one embodiment of the invention, the lipoxin A₄ analogs useful in treating or preventing intestinal fibrosis are selected from the compounds of formula (I).

In another embodiment, the lipoxin A₄ analogs useful in the present invention are selected from the compounds of formula (II).

In another embodiment of the invention, the lipoxin A₄ analogs useful in treating or preventing intestinal fibrosis are selected from the compounds of formula (II-a):

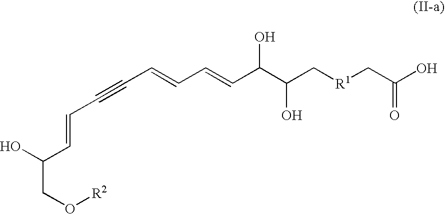

wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy); as a single stereoisomer, a mixture of stereoisomers, or a racemic mixture of stereoisomers; or as a cyclodextrin clathrate thereof, or as a pharmaceutically acceptable salt thereof.

In another embodiment of the invention, the lipoxin A₄ analog is 2-((2S,3R,4E,6E,10E,12S)-13-(4-fluorophenoxy)-2,3,12-trihydroxytrideca-4,6,10-trien-8-ynyloxy)acetic acid or a pharmaceutically acceptable salt thereof.

The pharmacologically active lipoxin A₄ analogs of formulas (I), (II) and (II-a) can be processed in accordance with the methods disclosed in U.S. Pat. No. 6,831,186 or by conventional methods of galenic pharmacy to produce medicinal agents for treating intestinal fibrosis. The pharmaceutical compositions comprise a lipoxin A₄ analog in a therapeutically effective amount (that is, an amount effective to treat or prevent intestinal fibrosis) and one or more pharmaceutically acceptable excipients. Suitable excipients may include, but are not limited to, pharmaceutical, organic or inorganic inert carrier materials suitable for enteral, parenteral or topical administration which do not deleteriously react with the active compounds. Suitable pharmaceutically acceptable carriers include, but are not limited to, water, salt solutions, alcohols, gelatine, gum arabic, lactate, starch, magnesium stearate, talc, vegetable oils, polyalkylene glycols, polyvinyl pyrrolidone, hydroxyl-methylcellulose, silicic acid, viscous paraffin, fatty acid monoglycerides and diglycerides, and the like. The pharmaceutical products may be in solid form, for example as tablets, coated tablets, suppositories or capsules, or in liquid form, for example as solutions, suspensions or emulsions. They may additionally comprise, where appropriate, auxiliary agents such as lubricants, preservatives, stabilizers, wetting agents, emulsifiers, salts to alter the osmotic pressure, buffers, coloring, flavoring, and/or aromatic substances and the like that do not deleteriously react with the active compounds. Examples of suitable pharmaceutical compositions include the following:

Particularly suitable for oral use are tablets, coated tablets or capsules with talc and/or carbohydrate carriers or binders, such as, for example, lactose, maize starch or potato starch. Use is also possible in liquid form, such as, for example, as fluid to which a sweetener is added where appropriate.

Sterile, injectable, aqueous or oily solutions are used for parenteral administration, as well as suspensions, emulsions or implants, including suppositories. Ampoules are convenient unit dosages. Sustained release compositions can be formulated including those wherein the active compound is protected with differentially degradable coatings, e.g., by microencapsulation, multiple coatings, etc.

Carrier systems which can also be used are surface-active excipients such as salts of bile acids or animal or vegetable phospholipids, but also mixtures thereof, and liposomes or constituents thereof. Transdermal patches may also be used as delivery means.

The dosage of the lipoxin A₄ analog will be that amount effective to treat or prevent intestinal fibrosis. The effective amount of active ingredient may vary depending on the route of administration, the age and weight of the patient, the nature and severity of the disorder to be treated, and similar factors. The effective amount can be determined by methods known to those of skill in the art. The daily dose is generally about 0.1-200 μg/kg/day, preferably about 0.5-10 μg/kg/day, when administered to human patients, it being possible for the dose to be given as a single dose to be administered once or divided into two or more daily doses.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil, for example.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include, but are not limited to, one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention.

Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable non-irritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The lipoxin A₄ analogs, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.7 mg) to about 100 mg/kg (i.e., 7.0 gm); preferably, a therapeutically effective dose is (for a 70 kg mammal) from about 0.01 mg/kg (i.e., 7 mg) to about 50 mg/kg (i.e., 3.5 gm); more preferably, a therapeutically effective dose is (for a 70 kg mammal) from about 1 mg/kg (i.e., 70 mg) to about 25 mg/kg (i.e., 1.75 gm). In general, one of ordinary skill in the art, acting in reliance upon personal knowledge and the disclosure of this Application, will be able to ascertain a therapeutically effective amount of a compound of Formula (I), (II) or (II-a) in U.S. Pat. No. 6,831,186 for treating a given disease.

Lipoxin A₄ analogs, or pharmaceutically acceptable derivatives thereof, may also be administered simultaneously with, prior to, or after administration of one or more other therapeutic agents for treating or preventing intestinal fibrosis. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

Without further elaboration, it is believed that one skilled in the art can, using the preceding description, utilize the present invention to its fullest extent. The following specific embodiments are, therefore, to be construed as merely illustrative, and not limitative of the remainder of the disclosure in any way.

### EXAMPLES

### In Vivo Experimental Studies

### Chronic Colitis Induction and Macroscopic Evaluation

### Material and Methods

A model of chronic inflammation induced by 7 week administration of 2,4,6-trinitrobenzenesulfonic acid (TNBS) in escalating manner was used in these studies. This model results in inflammation-driven fibrosis that associates with significant increase in intestinal content of collagen.

The induction of chronic colitis was performed according the protocol proposed by Lawrance et al, Gastroenterology (2003) 125:1750-1761. Six- to eight-week-old female mice of the BALB/c strain were obtained from Charles River (Breeding Laboratories, Monza, Italy).

In the below Studies, the lipoxin A₄ analog sodium 2-((2S,3R,4E,6E,10E,12S)-13-(4-fluorophenoxy)-2,3,12-trihydroxytrideca-4,6,10-trien-8-ynyloxy)acetate:

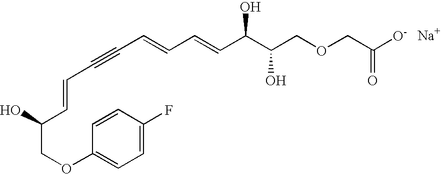

was used as the test compound. However, it is understood that this is only for illustrative purposes and that the other pharmaceutically effective lipoxin A₄ analogs of formulas (I), (II) and (II-a) are encompassed within this invention.

### Study 1 Protocol:

In a first in vivo experiment, BALB/c mice of the control group (n=7) received weekly 0.1 mL of saline solution by intracolonic injections. Mice of the TNBS group (n=15) were treated for 8 weeks with escalating doses (0.5 to 1.25 mg, with a 0.25 mg increase every two weeks) of TNBS in 30% ethanol by intracolonic injections at weekly intervals. Mice of TNBS+lipoxin A₄ analog group (n=15) received the same weekly TNBS administration as the TNBS-only group plus 1 mg/kg of the lipoxin A₄ analog daily by intragastric administration. Mice were examined weekly for weight loss, diarrhea and rectal bleeding. Two days after the last administration of TNBS, mice were killed and colons were removed and examined. The macroscopic appearance was analyzed considering the presence of indurations, edema, thickness and evidence of mucosal hemorrhage.

### Study 2 Protocol:

To test the influence of lipoxin analogs in the inflammatory and fibrotic response in animals with established colitis, in a second in vivo study lipoxin A₄ analog (1 mg/kg) was dosed orally starting after the third week of administration of TNBS (same protocol as described above). Mice were examined weekly for weight loss, diarrhea and rectal bleeding. Two days after the last administration of TNBS, mice were killed and colons were removed and examined. The macroscopic appearance was analyzed considering the presence of indurations, edema, thickness and evidence of mucosal hemorrhage.

### MPO Assays:

Neutrophil infiltration in the colon was monitored by measuring MPO activity using a spectrophotometric assay with trimethylbenzidine (TMB) as a substrate according to a previously published method. Activity is expressed as U per mg protein.

### Colon Histology:

For histologic examination, sections of the proximal, middle and distal colon from each animal were fixed in 10% formalin, embedded in paraffin, sectioned, and stained with haematoxylin and eosin (H&E) or Sirius Red. For the latter, sections were incubated for 30 minutes in 0.1% Sirius Red F3B containing saturated picric acid and 0.1% fast green. After rising twice with distilled water, sections were briefly dehydrated with 70% ethanol.

Images from colon sections were acquired with a BX60 microscope (Olympus Co., Rome, Italy), digitalized using a SPOT-2 camera (Diagnostic Instruments Inc., Sterling Heights, Mich.) with a resolution of 1315×1033 pixels and analyzed using a computerized image analysis system (Image Acquisition System Ver.005, Delta Sistemi, Rome, Italy).

Sections from the colon of each animal were examined in a blinded fashion. The degree of the inflammation and fibrosis was scored accordingly with an established method (Lawrance et al., supra; Neurath et al., J. Exp. Med. (1995) 182:1281-1290). Briefly, inflammation was scored as absent, mild, moderate or severe depending on the density and extent of inflammatory infiltrates, loss of goblet cells, and bowel wall thickening. Also, the fibrosis was scored as absent, mild, moderate or severe on the basis of the density and extent of the Sirius red-positive connective tissue staining, compared with the water-control mice.

### Gene Expression in Colon Tissue and Intestinal Fibroblasts:

Quantification of the expression of mouse gene in colon tissues and intestinal fibroblasts was performed by quantitative real-time polymerase chain reaction (RT-PCR) (Heid C A 1996), using sense and antisense primers indicated in Table 1. All PCR primers were designed using PRIMER3-OUTPUT software using published sequence data from the NCBI database. Total RNA was isolated (TRIzol reagent, Invitrogen srl, Milan, Italy) from specimens taken from distal colons and intestinal fibroblasts. One microgram of purified RNA was treated with Dnasel for 15 minutes at room temperature, followed by incubation at 95° C. for 5 minutes in the presence of 2.5 mmol/L EDTA. The RNA was reverse-transcribed with Superscript III (Invitrogen srl, Milan, Italy) in 20 μL reaction volume using random primers. For quantitative RT-PCR, 100 ng template was dissolved in a 25 μL containing 0.3 μmol/L of each primer and 12.5 μL of 2XZ SYBR Green PCR Master mix (Bio-Rad, Hercules, Calif.). All reactions were performed in triplicate, and the thermal cycling conditions were as follows: 2 minutes at 95° C., followed by 50 cycles of 95° C. for 10 seconds, and 60° C. for 30 seconds in an icycler iQ instrument (Bio-Rad, Hercules, Calif.). The mean value of the replicates for each sample was calculated and expressed as the cycle threshold (CT; cycle number at which each PCR reaction reaches a predetermined fluorescent threshold, set within the linear range of all reactions). The amount of gene expression was then calculated as the difference (.CT) between the CT value of the sample for the target gene and the mean CT value of that sample for the endogenous control (GAPDH). Relative expression was calculated as the difference (..CT) between .CT values of the test control sample for each target gene. The relative expression level was expressed as 2-..CT.

TABLE 1
Quantitative RT-PCR Primers

| Gene | Sense primer | Antisense primer |
|---|---|---|
| Mouse | 5′-ctgagtatgtcgtggagtctac-3′ | 3′-gttggtggtgcaggatgcattg-5′ |
| GAPDH | | |
| Mouse | 5′-acggcatggatctcaaagac-3′ | 3′-gtgggtgagcacgtagt-5′ |
| TNFalfa | | |
| Mouse | 5′-ttgcttcagctccacagaga-3′ | 3′-tggttgtagagggcaaggac-5′ |
| TGFbeta1 | | |
| Mouse α1(I) | 5′-acgtcctggtgaagttggtc-3′ | 3′-cagggaagcctctttctcct-5′ |
| collagen | | |

### Results

The two Studies above illustrate that a stable analog of lipoxin A₄ is effective in protecting against fibrosis development in chronic model of colon inflammation. When administered to mice for seven weeks, it significantly attenuated colon fibrosis development as measured by morphometric analysis of Sirius red-stained colon and markers of colon fibrosis, including colon expression of TGFβ, α-SMA, αI collagen, and fibronectin mRNA. In addition, modulation exerted by a lipoxin A₄ analogy on fibrosis markers was confirmed at protein level, since TGBβ protein was found to be reduced in mice administered lipoxin A₄ analog in comparison to mice administered TNBS alone. In addition to this effect, lipoxin A₄ analog administered orally attenuated colon inflammation.

Further, the results of the second Study confirmed that a lipoxin A₄ analog is also effective when administered in a therapeutic manner, i.e. starting on week 3 after TNBS administration.

What is claimed is:

1. A method of treating intestinal fibrosis in a patient in need of such treatment comprising administering to the patient a therapeutically effective amount of a lipoxin A₄ analog compound of formula (I) or formula (II): 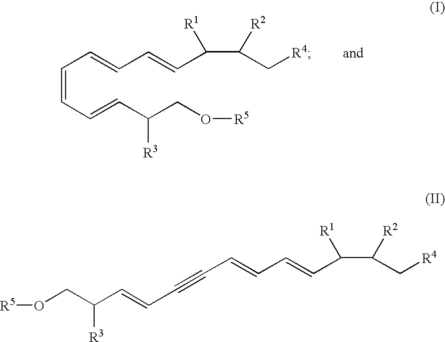
wherein:
each R¹, R² and R³ are independently halo, —OR⁶, —SR⁶, —S(O)ₜR⁷ (where t is 1 or 2) or —N(R⁷)R⁸;
or R¹ and R² together with the carbons to which they are attached form a monocyclic heterocyclic structure selected from the following: 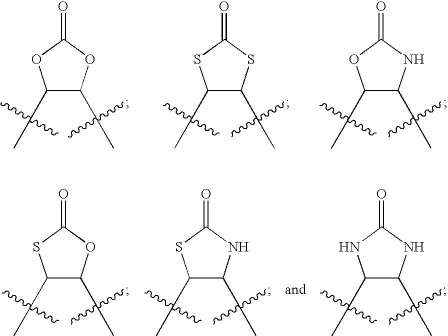
or R¹ and R² together with the carbons to which they are attached form the following bicyclic heterocyclic structure: 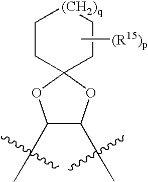
(where q is 0 to 3, p is 1 to 4 and each R¹⁵ is hydrogen, alkyl, aralkyl or aryl);
each R⁴ is —R⁹—R¹², —R⁹—R¹³—R¹¹, —R⁹—O—R¹⁰—R¹¹, —R⁹—O—R¹², —R⁹—C(O)—R¹⁰—R¹¹, —R⁹—N(R⁷)—R¹⁰—R¹¹, —R⁹—S(O)ₜR¹⁰—R¹¹ (where t is 0 to 2), or —R⁹—C(F)₂—R⁹—R¹¹;
each R⁵ is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
each R⁶ is independently hydrogen, alkyl, aryl, aralkyl, —C(O)R⁷, —C(S)R⁷, —C(O)OR¹⁴, —C(S)OR¹⁴, —C(O)N(R⁷)R⁸, or —C(S)N(R⁷)R⁸;
each R⁷ is independently hydrogen, alkyl, cycloalkyl, aryl, or aralkyl;
R⁸ is independently hydrogen, alkyl, aryl, aralkyl, —C(O)R⁷, —C(O)OR¹⁴, or cycloalkyl (optionally substituted with one more substituents selected from alkyl, —N(R⁷)₂, and —C(O)OR⁷);
each R⁹ is independently a direct bond or a straight or branched alkylene chain;
each R¹⁰ is independently a straight or branched alkylene chain, a straight or branched alkenylene chain, a straight or branched alkynylene chain or a cycloalkylene;
each R¹¹ is independently —C(O)OR⁷, —C(O)N(R⁷)₂, —P(O)(OR⁷)₂, —S(O)₂OR⁷, —S(O)₂N(H)R⁷ or tetrazole;
R¹² is aryl (substituted by —C(O)OR⁷ or —C(O)N(R⁷)₂ and optionally by one or more substituents selected from alkyl, alkoxy, halo, haloalkyl and haloalkoxy) or aralkyl (substituted by —C(O)OR⁷ or —C(O)N(R⁷)₂ and optionally by one or more substituents selected from alkyl, alkoxy, halo, haloalkyl and haloalkoxy);
R¹³ is a branched alkylene chain, a straight or branched alkenylene chain or a cycloalkylene; and
R¹⁴ is alkyl, aryl or aralkyl;
as a single stereoisomer, a mixture of stereoisomers, or a racemic mixture of stereoisomers;
or as a cyclodextrin clathrate thereof, or as a pharmaceutically acceptable salt thereof.

2. A method according to claim 1 wherein the lipoxin A₄ analog is selected from the compounds of formula (I).

3. A method according to claim 1 wherein the lipoxin A₄ analog is selected from the compounds of formula (II).

4. A method according to claim 3 wherein the lipoxin A₄ analog is selected from the compounds of formula (II-a): 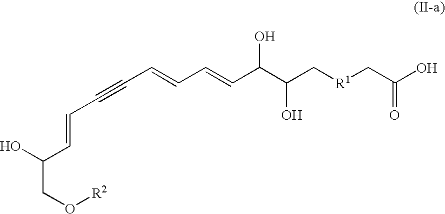
wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
as a single stereoisomer, a mixture of stereoisomers, or a racemic mixture of stereoisomers;
or as a cyclodextrin clathrate thereof, or as a pharmaceutically acceptable salt thereof.

5. A method according to claim 4 wherein the lipoxin A₄ analog Is 2-((2S,3R,4E,6E,10E,12S)-13-(4-fluorophenoxy)-2,3,12-trihydroxytrideca-4,6,10-trien-8-ynyloxy)acetic acid or a pharmaceutically acceptable salt thereof.

6. A method according to claim 5 wherein the lipoxin A₄ analog is sodium 2-((2S,3R,4E,6E,10E,12S)-13-(4-fluorophenoxy)-2,3,12-trihydroxytrideca-4,6,10-trien-8-ynyloxy)acetate.

7. A method of preventing intestinal fibrosis in a patient in need of such treatment comprising administering to the patient a therapeutically effective amount of a lipoxin A₄ analog compound of formula (I) or formula (II): 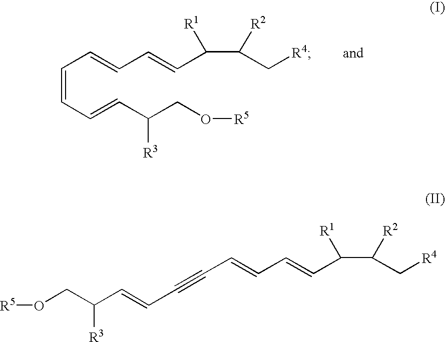
wherein:
each R¹, R² and R³ are independently halo, —OR⁶, —SR⁶, —S(O)ₜR⁷ (where t is 1 or 2) or —N(R⁷)R⁸;
or R¹ and R² together with the carbons to which they are attached form a monocyclic heterocyclic structure selected from the following: 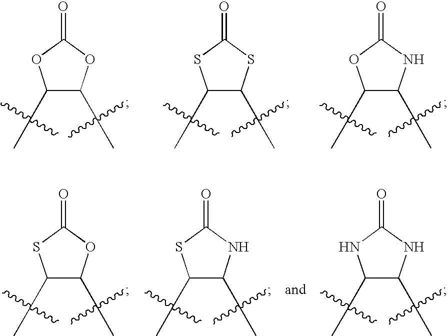
or R¹ and R² together with the carbons to which they are attached form the following bicyclic heterocyclic structure: 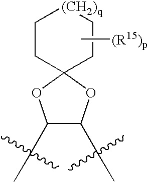
(where q is 0 to 3, p is 1 to 4 and each R¹⁵ is hydrogen, alkyl, aralkyl or aryl);
each R⁴ is —R⁹—R¹², —R⁹—R³—R¹¹, —R⁹—O—R¹⁰—R¹¹, —R⁹—O—R¹², —R⁹—C(O)—R¹⁰—R¹¹, —R⁹—N(R⁷)—R¹⁰—R¹¹, —R⁹—S(O)ₜR¹⁰—R¹¹ (where t is 0 to 2), or —R⁹—C(F)₂—R⁹—R¹¹;
each R⁵ is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
each R⁶ is independently hydrogen, alkyl, aryl, aralkyl, —C(O)R⁷, —C(S)R⁷, —C(O)OR¹⁴, —C(S)OR¹⁴—C(O)N(R⁷)R⁸, or —C(S)N(R⁷)R⁸;
each R⁷ is independently hydrogen, alkyl, cycloalkyl, aryl, or aralkyl;
R⁸ is independently hydrogen, alkyl, aryl, aralkyl, —C(O)R⁷, —C(O)OR¹⁴, or cycloalkyl (optionally substituted with one more substituents selected from alkyl, —N(R⁷)₂, and —C(O)OR⁷);
each R⁹ is independently a direct bond or a straight or branched alkylene chain;
each R¹⁰ is independently a straight or branched alkylene chain, a straight or branched alkenylene chain, a straight or branched alkynylene chain or a cycloalkylene;
each R¹¹ is independently —C(O)OR⁷, —C(O)N(R⁷)₂, —P(O)(OR⁷)₂, —S(O)₂OR⁷, —S(O)₂N(H)R⁷ or tetrazole;
R¹² is aryl (substituted by —C(O)OR⁷ or —C(O)N(R⁷)₂ and optionally by one or more substituents selected from alkyl, alkoxy, halo, haloalkyl and haloalkoxy) or aralkyl (substituted by —C(O)OR⁷ or —C(O)N(R⁷)₂ and optionally by one or more substituents selected from alkyl, alkoxy, halo, haloalkyl and haloalkoxy);
R¹³ is a branched alkylene chain, a straight or branched alkenylene chain or a cycloalkylene; and
R¹⁴ is alkyl, aryl or aralkyl;
as a single stereoisomer, a mixture of stereoisomers, or a racemic mixture of stereoisomers;
or as a cyclodextrin clathrate thereof, or as a pharmaceutically acceptable salt thereof.

8. A method according to claim 7 wherein the lipoxin A₄ analog is selected from the compounds of formula (I).

9. A method according to claim 7 wherein the lipoxin A₄ analog is selected from the compounds of formula (II).

10. A method according to claim 9 wherein the lipoxin A₄ analog is selected from the compounds of formula (II-a): 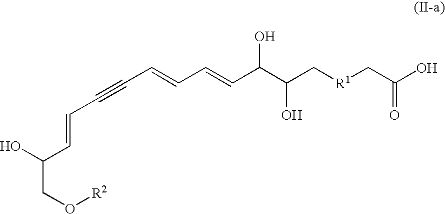
wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
as a single stereoisomer, a mixture of stereoisomers, or a racemic mixture of stereoisomers;
or as a cyclodextrin clathrate thereof, or as a pharmaceutically acceptable salt thereof.

11. A method according to claim 10 wherein the lipoxin A₄ analog is 2-((2S,3R,4E,6E,10E,12S)-13-(4-fluorophenoxy)-2,3,12-trihydroxytrideca-4,6,10-trien-8-ynyloxy)acetic acid or a pharmaceutically acceptable salt thereof.

12. A method according to claim 11 wherein the lipoxin A₄ analog is sodium 2-((2S,3R,4E,6E,10E,12S)-13-(4-fluorophenoxy)-2,3,12-trihydroxytrideca-4,6,10-trien-8-ynyloxy) acetate.
